(19) United States
(12) Patent Application Publication
(10) Pub. No.: US 2010/0069746 A1
(43) Pub. Date: Mar. 18, 2010

(54) FIDUCIAL MARKER PLACEMENT

(75) Inventor: Anthony St. John, Powys (GB)

Correspondence Address: EGBERT LAW OFFICES, 412 MAIN STREET, 7TH FLOOR, HOUSTON, TX, 77002, US

(73) Assignee: PROSURGICS LIMITED, BRACKNELL (GB)

(21) Appl. No.: 12/447,632

(22) PCT Filed: Oct. 24, 2007
(86) PCT No.: PCT/GB07/04065

§ 371 Date: Sep. 18, 2009

(30) Foreign Application Priority Data
Oct. 31, 2006 (GB) 0621705.3

(51) Int. Cl. A61B 5/05

(52) U.S. Cl. 600/426

(57) ABSTRACT

A method of determining the prospective location of two or more fiducial markers includes the steps of: with respect to the work piece, defining a path having a radius from an origin which varies in a non-repetitive manner with respect to the angular displacement of the path from the origin; and selecting two or more prospective locations of respective fiducial markers. The prospective locations are positioned relative to the work piece substantially at points along the defined path.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT Not applicable.

### REFERENCE TO AN APPENDIX SUBMITTED ON COMPACT DISC

Not applicable.

### BACKGROUND OF THE INVENTION

1. Field of the Invention

The present invention relates to a method of arranging fiducial markers on an object. In particular, embodiments of the present invention relates to the placement of fiducial markers on a work piece such as a medical patient.

2. Description of Related Art Including Information Disclosed Under 37 CFR 1.97 and 37 CFR 1.98.

When a work piece is to be acted upon it is sometimes necessary to register the actual location of the work piece with images thereof to ensure that any work is carried out on a correct region of the work piece. For example, an image of the internal structure of the work piece may be acquired and used as a guide when work is carried out on a part of the internal structure of the work piece which is not externally visible.

In such instances the frame of reference used to acquire the images of the work piece must be matched with the current frame of reference such that it is possible to direct a tool on or in the work piece to act upon an area of interest (such as part of the internal structure of the work piece). The tool may be directed utilizing images of the work piece which were acquired earlier; however, directing a tool in this manner is difficult because the current orientation of the work piece is usually different to the orientation of the work piece when the earlier images were acquired. In addition, the format of the images may not be conducive to such work. For example, image slices of a work piece may depict the work piece in its current orientation but directing a tool based upon the image slices may not be an easy procedure.

Generally, in order to register images of a work piece with the actual location of the work piece it is necessary to utilize features of the work piece which are visible in both the image and in the current view of the work piece.

For example, an x-ray image of the internal structure of a work piece must be interpreted with reference the external features of the work piece which are also visible in the x-ray image in order to achieve registration. However, the matching of images with actual features is difficult due to various different orientations of the features which are possible even when the features are matched in space.

Similarly, when a robot is to act on a work piece it is necessary for the precise orientation and position of the work piece to be determined within the spatial frame of reference of the robot. If the position and orientation of the work piece is not defined within the frame of reference of the robot then, for example, the robot cannot accurately align a work tool with the work piece in order to perform a task.

In some situations where robots are utilized in order to perform tasks on work pieces, the robot is programmed to operate on a work piece of a precisely known size and shape. The work piece is presented to the robot at a pre-determined position relative to the robot and, thus, the work piece is defined within the spatial frame of reference of the robot. For example, automated motor vehicle assembly lines include arrangements to place, for example, a motor vehicle at a known position relative to the robot. The size and shape of the motor vehicle is standard and this information has been programmed into the robot. The robot can, therefore, carry out a sequence of tasks in relation to the vehicle.

On the other hand, if the work piece is not in a predetermined position relative to the robot or the work piece is not of known dimensions (or both) then it is necessary for the precise position, orientation and dimensions of the work piece to be determined within the frame of reference of the robot before the work robot can perform any tasks on the work piece.

Additional problems with the definition of a work piece within the frame of reference of a robot occur when the work piece differs marginally from a sample work piece upon which the programming of the robot was carried out. Moreover, in certain situations, the work piece may move within the frame of reference of the robot during the tasks which are being performed by the robot on the work piece. Thus, although the work piece may have been defined within the spatial frame of the robot when a task was begun, by the time the task nears completion, the work piece may no longer be accurately defined within the frame of reference of the robot.

In order to overcome these problems fiducial markers have been developed.

Using fiducial markers it is possible to acquire images of the work piece including the fiducial markers and to match these images with, for example, internal scans of the work piece using methods such as computed tomography (CT), magnetic resonance (MR) and ultrasound imaging.

These markers can be adhered to the outside of the work piece or securely embedded within the work piece such that at least a portion of the marker is visible or otherwise detectable from the outside of the work piece. In some instances a frame including fiducial markers is attached to the work piece. The frame may be secured to the work piece using a number of pins which contact the work piece or which are embedded into the work piece. The use of a frame allows a number of fiducial markers to be securely located in fixed positions relative to the work piece using very few actual points of contact with the work piece. Thus, potential damage to the work piece is reduced. Furthermore, it is possible to adjust the position of the fiducial markers on the frame without the need to detach the markers from, and re-attach the markers to, the actual work piece.

In the majority of instances the fiducial markers (or frame) will be removed from the work piece after the robot has carried out its task or a series of tasks, or after the images have been matched with the work piece for whatever purpose may be relevant.

An example of the use of fiducial markers is in the medical scanning of a patient's brain prior to a medical operation or simply for diagnosis purposes. The fiducial markers may be adhered to the skin of a patient's head by means of an adhesive or adhesive tape. Alternatively, the fiducial markers can be embedded within the skull of the patient to ensure that they do not move or become loose. As will be appreciated, embedding fiducial markers in the skull of a patient is relatively expensive and can be uncomfortable for the patient. In general, embedded fiducial markers are only practical in situations where movement or loss of an adhered marker would induce significant problems which outweigh the detrimental effects of the embedded fiducial markers. The use of frames, as described above, is preferred as it ensures secure placement of the markers with respect to the patient without the need for large numbers of individually embedded fiducial markers.

The problems associated with both embedded and adhered fiducial markers will be explained below by way of an example.

Prior to surgery to remove a brain tumor from a patient's brain (or, for example, to insert electrodes into a patient's brain for the treatment of Parkinson's disease or in the study of epilepsy) it is necessary to take a number of images of the brain using, for example, CT x-ray scans, MR image scans or ultrasound scans. These images of the brain allow a surgeon to identify the position of the brain tumor (or other areas of interest) and sections of the brain which need to be removed (or where electrodes must be placed) during a subsequent operation.

On carrying out the actual operation, the surgeon ideally wishes to cause as little damage as possible to the healthy skull and tissue of the patient whilst still ensuring that as much as possible of the brain tumor is removed (or that the electrodes placed in the correct locations).

Therefore, it is necessary to provide a system to match the scanned images of the brain with the external view of the patient's head. In order to achieve this fiducial markers are placed on the patient's skull. A robot can be utilized to take a number of external images (e.g. two) of the patient's skull including the fiducial markers and these can be used to match the orientation of the patient's head with the images of the brain which were previously acquired, thus registering the patient's head with respect to a commonly defined frame of reference.

During optically image guided surgery, a frame including a number of fiducial markers may be secured to the patient (for example, to their head). MRI or CT x-ray images of a patient, the frame and the markers may be made to identify an area of interest within the patient. During a later surgical operation a tool is used to perform part of the surgical operation; the tool may include additional fiducial markers and/or may be attached to the frame. Two cameras are located such that they are operable to take images of the patient, the frame with fiducial markers and the fiducial markers on the tool (if there are any). The images of the patient are utilized to direct use of the tool (which may be manual—by a surgeon—or automatic—by a robot) with reference to the fiducial markers on the frame, the markers on the tool (if there are any), and the previously acquired images (eg. MRI or CT x-ray images). Thus the tool may be directed to the correct location without unnecessary damage to surrounding brain tissue.

It will be appreciated, that if any of the fiducial markers move or are rendered unusable for any reason, then it may not be possible to match accurately the external images of the patient's head with the internal images of the brain. This can result in a number of problems, for example, removal of healthy brain tissue during an operation.

Other problems occur due to the symmetry of an arrangement of fiducial markers on a work piece. For example, a square pattern of fiducial markers will yield four superficially valid orientations of the work piece within the frame of reference of a robot. To a certain extent, obviously symmetrical patterns can be avoided during placement of the markers. However, a pattern which appears to be asymmetrical from one view may exhibit symmetry in another view. Furthermore, if one or more of the markers becomes unusable, then the remaining markers may form a symmetrical pattern.

It is an object of the present invention to ameliorate the problems associated with the prior art.

### BRIEF SUMMARY OF THE INVENTION

Accordingly an aspect of the present invention provides a method of determining the prospective location of two or more fiducial markers comprising the steps of:
with respect to a work piece, defining a path having a radius from an origin which varies in a non-repetitive manner with respect to the angular displacement of the path from the origin; and
selecting two or more prospective locations of respective fiducial markers such that the prospective locations are positioned relative to the work piece substantially at points along the defined path.

Preferably, the prospective locations of the fiducial markers are substantially such that the angular displacement of any one prospective location from an adjacent prospective location along the path is not a co-prime of 360°.

Conveniently, the path is a Fibonacci or golden spiral.

Alternatively, the path is a logarithmic spiral.

Preferably, four or more prospective locations are selected such the position of the work piece can be determined with rotational invariance by studying the positions of the prospective locations.

Conveniently, the prospective locations of the fiducial markers are on the work piece.

Advantageously, the prospective locations are on one or more surfaces located at a fixed position with respect to the work piece.

Another aspect of the present invention provides a method of placing two or more fiducial markers onto a work piece comprising the steps of: defining a path with respect to the work piece, the path having a radius from an origin which varies in a non-repetitive manner with respect to the angular displacement of the path from the origin; selecting two or more prospective locations of respective fiducial markers such that the prospective locations are at positions relative to the work piece substantially at points along the defined path; and placing a fiducial marker substantially at each of the two or more prospective locations.

Advantageously, the prospective locations of the fiducial markers are substantially such that the angular displacement of any one prospective location from an adjacent prospective location along the path is not a co-prime of 360°.

Preferably, the path is a Fibonacci or golden spiral.

Alternatively, the path is a logarithmic spiral.

Advantageously, the method further comprises the step of securing the fiducial markers to the work piece substantially at each of the two or more prospective locations.

Preferably, securing comprises the step of embedding the fiducial markers in the work piece substantially at each of the two or more prospective locations.

Conveniently, securing comprises the step of adhering the fiducial markers to the work piece substantially at each of the two or more prospective locations.

Advantageously, the method further comprises the step of securing the fiducial markers to one or more surfaces located at a fixed position with respect to the work piece.

Preferably, fiducial markers are placed at four or more prospective locations such the position of the work piece can be determined with rotational invariance by studying the positions of the fiducial markers.

Another aspect of the present invention provides, a work piece on which two or more fiducial markers have been placed such that the markers are located substantially on a path defined with respect to the work piece, the path having a radius from an origin which varies in a non-repetitive manner with respect to the angular displacement of the path from the origin.

Preferably, the locations of the fiducial marker are substantially such that the angular displacement of any one location from an adjacent location along the path is not a co-prime of 360°.

Advantageously, the path is a logarithmic spiral.

Alternatively, the path is a Fibonacci or golden spiral.

Conveniently, the markers are secured to the work piece.

Preferably, the markers are embedded into the work piece.

Advantageously, the markers are adhered to the work piece.

Conveniently, four or more fiducial markers have been placed on the work piece such the position of the work piece can be determined with rotational invariance by studying the positions of the fiducial markers.

Another aspect of the present invention provides a computer programmed to carrying out the above method.

Another aspect of the present invention provides a program for operating a computer according to the above method.

### BRIEF DESCRIPTION OF THE SEVERAL VIEWS OF THE DRAWINGS

In order that the present invention may be more readily understood, embodiments thereof will be described, by way of example, with reference to the accompanying drawings.

FIG. 1 shows a schematic view of a work piece including a number of fiducial markers.

FIG. 2 shows a schematic view of medical patient's head including a number of fiducial markers.

FIG. 3 shows a schematic view of an approximation of a logarithmic spiral.

### DETAILED DESCRIPTION OF THE INVENTION

As will be appreciated from the above description of the problems associated with the prior art, the random placement of fiducial markers will not guarantee that there is no ambiguity in the orientation or position of a work piece when images of the work piece are matched with the work piece in three dimensional space. Thus, embodiments of the present invention provide methods of determining the position in which fiducial markers should be placed on a work piece such that, if some of the markers are rendered unusable (for example, they fall off, move or are occluded) the images of the work piece may still be accurately matched to the actual work piece or other images of the work piece.

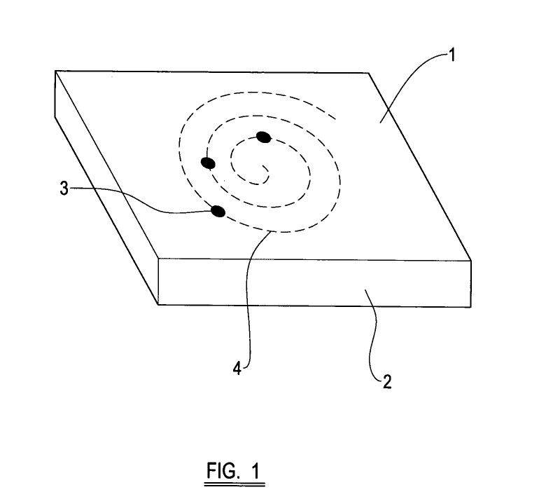

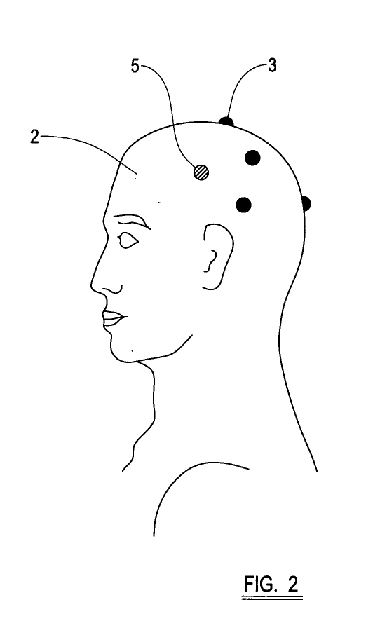

The present invention will be described with reference to FIGS. 1 and 2. In accordance with an embodiment of the present invention, if the surface 1 of a work piece 2 is flat (or substantially flat), then the fiducial markers 3 may be placed on a mathematical spiral 4 projected onto the surface 1, at intervals that follow a non-repeating pattern or sequence.

The markers 3 are placed along a spiral 4 which has a non-repetitive sequence of changes of radius for constant changes of angle from an origin.

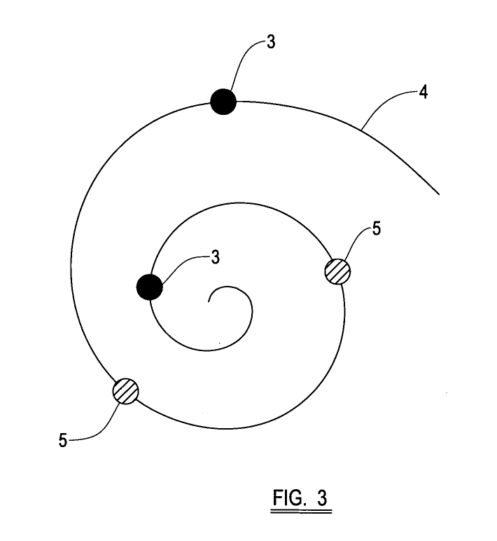

For example, the markers 3 could be placed on a logarithmic or equiangular (see Equation 1 and FIG. 3) spiral 4 having the following general polar equation:

r=a.e^{(bθ)} [Equation 1]

where r=radius, a,b=constants and θ=angle.

The constant “a” represents a scaling factor and the constant “b” determines how tightly and in which direction the spiral is plotted. The angular displacement of a point from the origin is represented by θ and will increase indefinitely until the radius of the spiral 4 is sufficient to encompass the work piece in question or the area of interest on the work piece 2.

A useful approximation to the logarithmic or equiangular spiral 4 can be achieved by using the Fibonacci spiral or the golden spiral. Either of these approximations can be used in accordance with embodiments of the present invention.

The logarithmic spiral and its approximations are suitable for the determination of the placement of fiducial markers 3 on a work piece 2 because for constant changes in angle, the radius increases at an increasing and non-repetitive manner.

A skilled person will appreciate that other equations will also meet the criteria given above for a spiral 4 that for constant changes in angle has a non-repetitive sequence of changes of radius. These other equations can be utilized in accordance with embodiments of the present invention.

If one of the above spirals 4 is projected onto the flat surface 1 of the work piece 2 and the fiducial markers 3 are adhered to the surface 1 of the work piece on the projected line of the spiral 4 at regular angular intervals, then scaling errors can occur which prevent images of the work piece 2 from being matched unambiguously to the actual work piece 2 (or other images thereof) if some of the markers 3 become unusable. This problem can be ameliorated by selecting angular separations for adjacent markers 3 which are not co-primes with 360°.

Thus, by adhering fiducial markers 3 to the flat surface 1 of the work piece 2 in accordance with the method described above it is possible to prevent misalignment (or misregistration) of the work piece 2 when images of the work piece 2 are matched to the work piece 2 itself or to other images or models of the work piece 2.

It is appreciated that most work pieces 2 will not comprise a flat surface 1 onto which a spiral 4 can be projected. Therefore, the present invention also provides for methods of determining prospective positions at which to locate markers 3 on a three dimensional shape.

According to one aspect of the invention, one of the above described spirals 4 is utilized and prospective marker locations 5 are determined in the manner also described above. The prospective locations 5 of the markers 3 are then projected onto a three dimensional work piece 2 and fiducial markers 3 placed at the intersections between the projected prospective mark locations 5 and a surface of the work piece 2. Of course, the locations of the markers 3 could equally be calculated after the spiral 4 is projected onto the work piece 2.

It will be understood that the above method of projecting the prospective locations 5 of markers 3 onto a three dimensional work piece 2 is similar to the stereographic projection utilized to produce maps, but in reverse. This becomes more apparent if the three dimensional work piece 2 is a section of a sphere.

According to other aspects of the present invention the equations used to calculate the spirals 4 described above are altered to include a depth coordinate (rather than simply operating in two dimensional space). Thus, the depth of the spiral 4 will also vary as a function of the angle.

Ideally the markers 3 should positioned in a non-planar arrangement to surround any areas of interest within the work piece 2; for example, in a medical patient the markers 3 may surround a potential surgical target such as a brain tumor.

Improved accuracy of image registration of alignment can be achieved if the fiducial markers 3 are arranged in a cluster such that they have a centroid that coincides with the area of interest within the work piece 2. In some cases this will not always be possible; therefore, some improvement in the accuracy of such a system can be achieved if the cluster of markers 3 is such that the any plane cutting through the area of interest also cuts through the marker cluster.

For example, the subthalamic nucleus is often an area of interest in the brain of medical patients. A cluster of markers 3 placed in accordance with aspects of the present invention and intended to optimize the accuracy of image registration or alignment in this area should be placed such that their centroid falls substantially over the subthalamic nucleus.

The spirals 4 and prospective marker locations 5 can be projected onto an actual work piece 2 using, for example, light projection equipment, or can be projected onto a virtual work piece. The virtual work piece may be created by acquiring two or more images of the work piece 2 from different angles and using the stereo image to form a virtual three dimensional model of the work piece 2.

In some embodiments, the prospective marker locations 5 can be determined using a virtual three dimensional model of the work piece 2 and then determining the prospective marker locations 5 on the actual work piece 2 by reference to features of the work piece 2 or additional temporary fiducial markers (not shown) which are used to ensure accurate placement of the actual fiducial markers 3 (the temporary markers may be removed after placement of the actual markers 3).

The fiducial markers 3 can be adhered to the actual work piece 2 or can be supported by one or more support structures (not shown), such as arms, in respective marker 3 locations relative to the work piece 2.

Indeed, in some embodiments the markers 3 are placed on a shell (not shown) in the form of a hollow dome or cone on the line of a spiral 4 as previously described. The shell is attached to a frame which may be secured to a work piece 2 (such as a patient). The shell may be moveable with respect to the frame such that it may be located (and securely fastened) over a particular area of interest.

For example, the work piece may be a patient's head and the shell may be placed such that the centroid of the markers 3 is located over an area of interest in the patient's brain.

Advantageously, tools may also be attached to the frame. Preferably, the tools include additional fiducial markers used for registering the location of the tools.

It will be appreciated that the shell could comprise a number of surfaces which need not be connected to each other. Indeed, the marker may be place on arms which extend from the frame. Alternatively, both a shell and arms could be utilized.

Thus, it will be appreciated that the aspects of the present invention can be utilized with the optically tracked image guided surgery method previously described.

Preferably, the work piece 2 is part of a human or animal body (for example, a medical patient). It will be appreciated that aspects of the present invention are suitable to application in the positioning of fiducial markers 3 on patient's head prior to or as a part of brain surgery, an investigation, or other treatment (such as radiotherapy or electrode placement).

It will be appreciated that when embodiments of the present invention are applied in practical situations the exact positioning of a fiducial marker 3 in accordance with the determined prospective marker location 5 may not be possible. However, the advantages of the present invention may still be achieved by using the prospective marker location 5 as an approximation for the actual location of the marker 3. Thus, the actual marker 3 may be placed close to the prospective marker location 5 and the advantages of the present invention will still be obtained.

As mentioned above, aspects of the present invention can be used for the purpose of alignment or registration of two or more images of a work piece 2, or the registration of a work piece 2 with the frame of reference of a robot (not shown). Aspects of the present invention allow such alignment or registration even when one or more of the markers 3 become unusable.

For example, fiducial markers 3 may be placed, in accordance with the method described above, on a work piece 2 which is, in this example, a patient's head. One or more images, for example, of the brain of the patient can be taken using a method such as MR imaging; the one or more images would include images of the locations of fiducial markers 3 on the patient's head. The MR images may be used, for example, to identify an area for surgery or further investigation. With the fiducial markers 3 in the same positions on the patient's head (preferably not having been removed in the interim period) the patient may be prepared for surgery. In this example, a robot is involved in the surgical process and the robot captures at least two images of the patient's head in order to construct a three dimensional model of the patient's head. The at least two images include images of the fiducial markers 3 on the patient's head. The robot can then be used to match, align or register the three dimensional model of the patient's head with the MR images of the brain which were acquired earlier. Thus, it is possible to determine the location of the area of surgical interest in the brain by viewing the patient's head with reference to the aligned images of the brain and model of the head of the patient.

It will be appreciated that the three dimensional model of the head may be acquired prior to the capturing of images of the brain and the model and images subsequently aligned. However, in such an example, the patient's head may no longer be registered within the spatial frame of reference of the robot and, thus, the use of the robot may be limited in that (without the reconstruction of the model from new images) the robot will not be able to determine the exact location of the patient's head within its frame of reference.

The above alignment, registration, or matching can still occur so long as there are at least four markers 3 on the work piece. Alternatively, any number of markers 3 may be utilized and one or more features of the work piece may be used as a virtual fiducial marker. Accurate alignment will be dependent upon there being a total of at least four fiducial 3 and virtual fiducial markers.

The advantages of the present invention are possible because the markers 3 are placed, in accordance with aspects of the present invention, at locations on the work piece 2 such that there is, for example, no symmetry between the locations of the fiducial markers 3—even if one or more of the markers become useable.

The term “path” as used in this specification and claims, includes both a curving line (or spiral) with no straight sections and a series of substantially straight lines which define an approximation to a spiral (or any combination of one or more curving lines and one or more straight lines).

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

1. A method of determining the prospective location of two or more fiducial markers, the method comprising the steps of:
defining a path with respect to a work piece, said path having a radius from an origin variable in a non-repetitive manner with respect to the angular displacement of the path from the origin; and
selecting two or more prospective locations of respective fiducial markers such that the prospective locations are positioned relative to the work piece substantially at points along the defined path.

2. A method according to claim 1, wherein the prospective locations of the fiducial markers are substantially such that the angular displacement of any one prospective location from an adjacent prospective location along the path is not a co-prime of 360°.

3. A method according to claim 1, wherein the path is a Fibonacci or golden spiral.

4. A method according to claims 1, wherein the path is a logarithmic spiral.

5. A method according to claim 1, wherein four or more prospective locations are selected such the position of the work piece can be determined with rotational invariance by studying the positions of the prospective locations.

6. A method according to claim 1, wherein the prospective locations of the fiducial markers are on the work piece.

7. A method according to claim 1, wherein the prospective locations are on one or more surfaces located at a fixed position with respect to the work piece.

8. A method of placing two or more fiducial markers onto a work piece, the method comprising the steps of:
defining a path with respect to the work piece, the path having a radius from an origin variable in a non-repetitive manner with respect to the angular displacement of the path from the origin;
selecting two or more prospective locations of respective fiducial markers such that the prospective locations are at positions relative to the work piece substantially at points along the defined path; and
placing a fiducial marker substantially at each of the two or more prospective locations.

9. A method according to claim 8, wherein the prospective locations of the fiducial markers are substantially such that the angular displacement of any one prospective location from an adjacent prospective location along the path is not a co-prime of 360°.

10. A method according to claim 8, wherein the path is a Fibonacci or golden spiral.

11. A method according to claim 8, wherein the path is a logarithmic spiral.

12. A method according to claim 8, further comprising the step of:
securing the fiducial markers to the work piece substantially at each of the two or more prospective locations.

13. A method according to claim 12, wherein the step of securing further comprises the step of:
embedding the fiducial markers in the work piece substantially at each of the two or more prospective locations.

14. A method according to claim 12, wherein the step of securing further comprises the step of:
adhering the fiducial markers to the work piece substantially at each of the two or more prospective locations.

15. A method according to claim 8, further comprising the step of:
securing the fiducial markers to one or more surfaces located at a fixed position with respect to the work piece.

16. A method according to claim 8, wherein fiducial markers are placed at four or more prospective locations such the position of the work piece can be determined with rotational invariance by studying the positions of the fiducial markers.

17. A workpiece system comprising:
a work piece having two or more fiducial markers placed such that the markers are located substantially on a path defined with respect to the work piece, the path having a radius from an origin which varies in a non-repetitive manner with respect to the angular displacement of the path from the origin.

18. A work piece system according to claim 17, wherein the locations of the fiducial marker are substantially such that the angular displacement of any one location from an adjacent location along the path is not a co-prime of 360°.

19. A work piece system according to claim 17, wherein the path is a logarithmic spiral.

20. A work piece system according to claim 17, wherein the path is a Fibonacci or golden spiral.

21. A work piece system according to claim 17, wherein the markers are secured to the work piece.

22. A work piece system according to claim 21, wherein the markers are embedded into the work piece.

23. A work piece system according to claim 21, wherein the markers are adhered to the work piece.

24. A work piece system according to claim 17, wherein four or more fiducial markers have been placed on the work piece such the position of the work piece can be determined with rotational invariance by studying the positions of the fiducial markers.
